SEQUENCE LISTING (23 sequences)

SEQ ID NO: 1 - DNA, 34 bp, Frankliniella occidentalis
ttagaattcgttgatacagccctgtttcatgtgc

SEQ ID NO: 2 - DNA, 32 bp, Frankliniella occidentalis
tttaagcttttagtcagtgcttgctctgcagc

SEQ ID NO: 3 - DNA, 20 bp, Frankliniella occidentalis
agcggataccgtagaagcac

SEQ ID NO: 4 - DNA, 20 bp, Frankliniella occidentalis
taggccggtaacggtaagtg

SEQ ID NO: 5 - DNA, 19 bp, Frankliniella occidentalis
aggatgctgctaacaacta

SEQ ID NO: 6 - DNA, 18 bp, Frankliniella occidentalis
gatgcggtccaatacaag

SEQ ID NO: 7 - DNA, 18 bp, Frankliniella occidentalis
ctccaggtcacagagatt

SEQ ID NO: 8 - DNA, 19 bp, Frankliniella occidentalis
caccaatgatgaggatagc

SEQ ID NO: 9 - DNA, 18 bp, Frankliniella occidentalis
gaaggattgacagattga

SEQ ID NO: 10 - DNA, 18 bp, Frankliniella occidentalis
tagagtctcgttcgttat

SEQ ID NO: 11 - DNA, 18 bp, Frankliniella occidentalis
ttgaagaagcatcctaac

SEQ ID NO: 12 - DNA, 18 bp, Frankliniella occidentalis
ttgagaagtagttgagat

SEQ ID NO: 13 - DNA, 2450 bp, Escherichia coli
acctgcggccgcaagcttggatccgaattcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccgagctcgcggccgcaagc

SEQ ID NO: 14 - DNA, 40 bp, Escherichia coli
gagctgttgacaattaatcatcggctcgtataatgtgtgg

SEQ ID NO: 15 - DNA, 138 bp, Artificial Sequence
  thrips cassette comprising PTAC sequence
cagctggagctgttgacaattaatcatcggctcgtataatgtgtgggaattcgagctcggatcctctagactcgagggaagcttccacacattatacgagccgatgattaattgtcaacagctcgatatcatcagctg

SEQ ID NO: 16 - DNA, 2588 bp, Artificial Sequence
  pEX-A-Thrips Cassette
acctgcggccgcaagcttggatccgaattcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccgagctcgcggccgcaagccagctggagctgttgacaattaatcatcggctcgtataatgtgtgggaattcgagctcggatcctctagactcgagggaagcttccacacattatacgagccgatgattaattgtcaacagctcgatatcatcagctg

SEQ ID NO: 17 - DNA, 316 bp, Frankliniella occidentalis
tctagagcagatgccatcagacaagacaattggaggaggtgatgattcttttaacacattcttcagcgagacgggcgctggcaaacacgtccccagggctgtctttgttgacctggaacccactgtagttgatgaggtccgcactggtacctaccgccagctcttccaccctgagcagcttatcaccggcaaggaggatgctgctaacaactatgctcgtggtcactacaccattggcaaggaaattgtcgaccttgtattggaccgcatccgcaagttggctgaccagtgcacaggtcttcaaggcttctctaga

SEQ ID NO: 18 - DNA, 400 bp, Frankliniella occidentalis
tctagaggttctcgacaagctgaaggctgagcgtgaacgtggtatcaccattgacattgctctctggaagttcgaaactccaaagtacaacgtcactgtcatcgatgctccaggtcacagagatttcattaagaacatgattactggtacctcccaagctgactgcgctatcctcatcattggtggtggtgttggtgaattcgaagctggtatctccaaggatggtcaaaccagagaacacgctcttctctctttcaccctcggtgtcagacaactcattgtcgccgtcaacaagatggacaccactcaatactctgaagcccgtttcactgaaattatcaaggaaacccaaaacttcatcaaggagggtcggttacaaccctaagacaccgcctctaga

SEQ ID NO: 19 - DNA, 2898 bp, Artificial Sequence
  pTub3
acctgcggccgcaagcttggatccgaattcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccgagctcgcggccgcaagccagctggagctgttgacaattaatcatcggctcgtataatgtgtgggaattcgagctcggatcctctagagcagatgccatcagacaagacaattggaggaggtgatgattcttttaacacattcttcagcgagacgggcgctggcaaacacgtccccagggctgtctttgttgacctggaacccactgtagttgatgaggtccgcactggtacctaccgccagctcttccaccctgagcagcttatcaccggcaaggaggatgctgctaacaactatgctcgtggtcactacaccattggcaaggaaattgtcgaccttgtattggaccgcatccgcaagttggctgaccagtgcacaggtcttcaaggcttctctagactcgagggaagcttccacacattatacgagccgatgattaattgtcaacagctcgatatcatcagctg

SEQ ID NO: 20 - DNA, 2982 bp, Artificial Sequence
  pElong1
acctgcggccgcaagcttggatccgaattcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccgagctcgcggccgcaagccagctggagctgttgacaattaatcatcggctcgtataatgtgtgggaattcgagctcggatcctctagaggttctcgacaagctgaaggctgagcgtgaacgtggtatcaccattgacattgctctctggaagttcgaaactccaaagtacaacgtcactgtcatcgatgctccaggtcacagagatttcattaagaacatgattactggtacctcccaagctgactgcgctatcctcatcattggtggtggtgttggtgaattcgaagctggtatctccaaggatggtcaaaccagagaacacgctcttctctctttcaccctcggtgtcagacaactcattgtcgccgtcaacaagatggacaccactcaatactctgaagcccgtttcactgaaattatcaaggaaacccaaaacttcatcaaggagggtcggttacaaccctaagacaccgcctctagactcgagggaagcttccacacattatacgagccgatgattaattgtcaacagctcgatatcatcagctg

SEQ ID NO: 21 - DNA, 722 bp, Artificial Sequence
  RNAseIII Bfo2
gttgatacagccctgtttcatgtgcaattaacagacgcaaagaataactggaacagcatgaaccccattcttataaataagctgcagcgtaaactaggctacacttttactcatccggaacttctgcaacaagccttgacgcatcgcagtgccagtagtaaacataatgaaagattagaatttcttggggattcaatcttaagctatgtcatcgcgaatgcgctttattgtcaattccctcgtgttgatgaaggggatatgagccggatgcgtgcgaccttggttagggggaatacccttgctgagatggcacgggagttcgaactgggtgagtgcttacgcctaggccctggtgagcttaagagtggtggatttcgccgtgaatccattttagcggataccgtagaagcactgattggcggaattttcttggatagcgatatccaacaaaccgaaaaattaatccttaattggtatcagtcgcggctggaagcgatcagcccgggtgataagcaaaaagatccgaaaacacgtttacaagagtatcttcaagggcgtcacttaccgttaccggcctatttagtggttcaggtgaggggagaggctcatgatcaagaatttacgattcactgccaagtgagtggtatgaatgaagcggtggttggcattggttctagccgtcgtaaagcagaacaggctgctgcagagcaagcactgactaa

SEQ ID NO: 22 - DNA, 3384 bp, Artificial Sequence
  pRNA1
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacagatctggggaattcgttgatacagccctgtttcatgtgcaattaacagacgcaaagaataactggaacagcatgaaccccattcttataaataagctgcagcgtaaactaggctacacttttactcatccggaacttctgcaacaagccttgacgcatcgcagtgccagtagtaaacataatgaaagattagaatttcttggggattcaatcttaagctatgtcatcgcgaatgcgctttattgtcaattccctcgtgttgatgaaggggatatgagccggatgcgtgcgaccttggttagggggaatacccttgctgagatggcacgggagttcgaactgggtgagtgcttacgcctaggccctggtgagcttaagagtggtggatttcgccgtgaatccattttagcggataccgtagaagcactgattggcggaattttcttggatagcgatatccaacaaaccgaaaaattaatccttaattggtatcagtcgcggctggaagcgatcagcccgggtgataagcaaaaagatccgaaaacacgtttacaagagtatcttcaagggcgtcacttaccgttaccggcctatttagtggttcaggtgaggggagaggctcatgatcaagaatttacgattcactgccaagtgagtggtatgaatgaagcggtggttggcattggttctagccgtcgtaaagcagaacaggctgctgcagagcaagcactgactaaaagcttggctgcagatctggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 23 - DNA, 5109 bp, Artificial Sequence
  pRNA2
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacagatctggggaattcgttgatacagccctgtttcatgtgcaattaacagacgcaaagaataactggaacagcatgaaccccattcttataaataagctgcagcgtaaactaggctacacttttactcatccggaacttctgcaacaagccttgacgcatcgcagtgccagtagtaaacataatgaaagattagaatttcttggggattcaatcttaagctatgtcatcgcgaatgcgctttattgtcaattccctcgtgttgatgaaggggatatgagccggatgcgtgcgaccttggttagggggaatacccttgctgagatggcacgggagttcgaactgggtgagtgcttacgcctaggccctggtgagcttaagagtggtggatttcgccgtgaatccattttagcggataccgtagaagcactgattggcggaattttcttggatagcgatagctttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccggctcacggtaactgatgccgtatttgcagtaccagcgtacggcccacagaatgatgtcacgctgaaaatgccggcctttgaatgggttcatgtgcagctccatcagcaaaaggggatgataagtttatcaccaccgactatttgcaacagtgccgttgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagagggcgggatgcgaagaatgcgatgccgctcgccagtcgattggctgagctcatgagcggagaacgagatgacgttggaggggcaaggtcgcgctgattgctggggcaacacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcataaagctatccaacaaaccgaaaaattaatccttaattggtatcagtcgcggctggaagcgatcagcccgggtgataagcaaaaagatccgaaaacacgtttacaagagtatcttcaagggcgtcacttaccgttaccggcctatttagtggttcaggtgaggggagaggctcatgatcaagaatttacgattcactgccaagtgagtggtatgaatgaagcggtggttggcattggttctagccgtcgtaaagcagaacaggctgctgcagagcaagcactgactaaaagcttggctgcagatctggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

(19) United States
(12) Patent Application Publication
(10) Pub. No.: US 2018/0195137 A1
(43) Pub. Date: Jul. 12, 2018

(54) PEST CONTROL SYSTEM

(71) Applicant: Swansea University, Swansea, South Wales (GB)

(72) Inventors: Paul Dyson, Swansea (GB); Miranda Whitten, Swansea (GB)

(73) Assignee: Swansea University, Swansea, South Wales (GB)

(21) Appl. No.: 15/580,042

(22) PCT Filed: Jun. 8, 2016
(86) PCT No.: PCT/GB2016/051683

§ 371 Date: Dec. 6, 2017

(30) Foreign Application Priority Data
Jun. 11, 2015 (GB) 1510154.6

(51) Int. Cl. C12R 1/01; C12N 15/113; C07K 16/10; A01N 63/02; A01N 37/46; C12N 15/74; A01N 25/00

(52) U.S. Cl. CPC C12R 1/01; C12N 15/113; C07K 16/1036; C12N 2310/14; A01N 37/46; C12N 15/746; A01N 25/006; A01N 63/02

(57) ABSTRACT

The invention relates to a genetically transformed or transfected bacterial cell of a gut symbiont of an insect belonging to the Order Thysanoptera wherein said cell is transformed to express double-stranded RNA (dsRNA) active against at least one selected insect gene; a vector for transforming or transfecting said bacterial cell; an insect including said transformed bacterial cell and a method of pest control employing the use of said bacterial cell and/or said insect.

### FIELD OF THE INVENTION

The invention relates to a genetically transformed or transfected bacterial cell that is a gut symbiont of an insect belonging to the Order Thysanoptera wherein said cell is transformed to express double-stranded RNA (dsRNA) active against at least one selected insect gene; a vector for transforming or transfecting said bacterial cell; an insect including said transformed bacterial cell and a method of pest control employing the use of said bacterial cell and/or said insect.

### BACKGROUND OF THE INVENTION

Invertebrates and other pests are common vectors for pathogenic organisms, typically micro-organisms that are responsible for a variety of diseases that can affect crops.

Over the past 30 years, insects such as western flower thrips (WFT), Frankliniella occidentalis (Pergande) (Thysanoptera: Thripidae), has become one of the most important agricultural pests worldwide. Its pest status can be attributed to several factors, including its reproductive potential, invasiveness (endemic to Western North America, it has invaded many countries in Europe, Africa and Asia), range of host crops, ability to transmit plant viruses, and insecticide resistance. All of these factors are interrelated, and are linked to the life cycle and life history strategy of the species. It is a significant pest of greenhouse and field crops and soft fruit in Europe, and staples such as beans, cowpeas and groundnuts in Africa and Asia and this can have devastating effects on humans and animals that rely on those crops for nutrition.

Direct crop damage results from both feeding and damage of plants by way of oviposition. Thrips also transmit several different tospoviruses, including Groundnut ringspot virus (GRSV) and Tomato spotted wilt virus (TSWV) and it has been estimated that TSWV alone causes over $1 billion in losses annually. Over 1,000 species of plants in 84 families are susceptible to TSWV, giving it one of the broadest host ranges of any plant pathogen.

Consequently, there is a continued interest in developing control methods aimed at reducing or eradicating the incidence of the above agricultural pests. This is often achieved either by targeting the pathogen itself (GRSV/TSWV) or the vector (WFT) commonly associated with transmission. Methods for controlling infestations and infection by insects have typically been in the form of; physical barriers preventing transmission; chemical compositions, such as insecticides, chemical drugs and repellents; and also biological controls.

Recent advances in genetics has led to a greater understanding of the development of a vast range of organisms, and paved the way for new avenues in biological pest control. The study of insect gene function provides a crucial step towards understanding the physiology, behaviour, immunology and even disease transmission in this very diverse and successful group of organisms. Armed with this knowledge it is possible to develop ways to fight crop damage and develop strategies to control pest insect populations.

RNA interference (RNAi) is a powerful technique of sequence-specific down-regulation of gene expression to interrogate eukaryotic gene function on an individual gene basis.

RNAi is a form of post-transcriptional gene silencing wherein a specific mRNA of a particular gene is destroyed or blocked, preventing translation and formation of an active gene product. RNAi occurs naturally within living cells to modulate gene activity, and is also important in defence against parasites and viral infection. For example, when a cell is injected with RNA in a double-stranded (ds) form, a protein called Dicer (or RNase III) cleaves the dsRNA molecules into short fragments of RNA (20-25 nucleotides), termed short interfering RNA (siRNA) due to their ability to interfere with the expression of a specific gene. These siRNA molecules are unwound into single stranded (ss) RNA, whereupon the so-called guide strand is incorporated into the RNA-induced silencing complex (RISC). Often, this guide strand will base pair with a complimentary sequence of mRNA in the cell inducing its cleavage by the catalytic component of the RISC complex. The mRNA is not translated and no functional protein is produced, and therefore the effects of the gene encoding the specific mRNA are ‘silenced’. This process is termed cell-autonomous RNAi, wherein gene silencing is limited to the cell in which the dsRNA is introduced. Alternatively, environmental and systemic RNAi are the two forms of non-cell autonomous RNAi, wherein the interfering effect takes place in cells/tissues different from where the dsRNA was introduced/produced. In this case, the dsRNA is either taken up into multiple cells (environmental RNAi such as in viral infections), or the silencing signal is transported from the cell in which the dsRNA is applied or expressed to other cells where the effect is observed (systemic RNAi).

By artificially synthesising dsRNA (or siRNA molecules) with a known sequence complimentary to a gene of interest, and introducing it to target cells, it is possible to understand the role of a specific gene by observing the consequences of its loss of activity. RNAi and other so-called reverse genetics techniques are thus revolutionizing biological sciences, with applications in genomics, biotechnology, and medicine.

RNAi in invertebrates is an established technology, wherein dsRNA is delivered most commonly by injection. However, this process often has high mortality rates due to injection trauma and anaesthesia, and also requires high sample numbers. Large insects also require expensive quantities of dsRNA to be synthesised. Moreover, as stated, this results in cell-autonomous gene silencing achieving transient RNAi effects and, therefore, is not applicable for the control of insect pests in the field. As a result, for efficient insect pest control non-cell autonomous RNAi is required, which has been shown to be achieved by feeding insects with a biological source such as genetically modified bacteria or plant material. This therefore begins with the uptake of dsRNA environmentally into the gut lumen of the insect, from where it spreads to tissues elsewhere (systemic RNAi). Achieving RNAi depends on a reliable method for delivering, or uptake of, a dsRNA copy of part of a target gene to the insect. For some insect species this has been achieved by including in their food live or dead E. coli cells expressing dsRNA. The bacteria are digested in the gut and the dsRNA is taken up and delivered systemically to different tissues where it can mediate transient RNAi. EP2374462A2 teaches that RNAi can be introduced by ingestion of: naked dsRNA, food contaminated with E. coli expressing dsRNA e.g. by spraying with transformed bacteria, or genetically modified plant material expressing dsRNA. WO2011017137A2 teaches a method whereby a food bait of the insect is contaminated with genetically modified bacteria expressing dsRNA and the bait is returned to a colony to be fed on by the insects. Further, WO2011025860A1 teaches the use of RNAi against plant-feeding insects, wherein bacteria that infect specific plants are genetically modified to express specific dsRNA. Similarly, WO2011036536A2 teaches specific RNAi gene targets which are silenced by the delivery of dsRNA by spraying dead bacteria onto crop plants.

However, many of the currently developed techniques are often impractical or have poor efficacy and are not targeted at organisms that cause particular harm to the environment. For example, contamination of food sources (plant or other) with genetically modified bacteria may be harmful toward other insects that may be occasional feeders. Furthermore, spraying chemical compositions containing naked dsRNA onto insects or their food source may also have implications on other non-pest organisms. More importantly, all of the existing methods only teach delivery methods whereby transient gene-silencing effects are observed. Many of the currently employed techniques only exhibit short silencing durations, which are often too transient for certain targets e.g. those for hormone and developmental studies. Therefore re-application of dsRNA (or other RNAi constructs) is often required, which is costly and time-consuming. Furthermore, as currently employed techniques are not transferrable, effective pest control has been difficult to demonstrate. Therefore the technology in its current state is inappropriate for many insect species and improved efficacy and methods of application are required.

We have therefore developed a new RNAi technique effective against insects belonging to the Order Thysanoptera that relies on the in vivo synthesis of dsRNA by transgenic symbiotic gut bacteria from a species that naturally reside in the insect. Moreover, we developed a new RNAi technique that kills the insects in both the larval and adult stage but particularly the larval stage thus, advantageously, before insect pest reproduction and feeding occurs and before transmission of plant pathogens between individual plants occurs.

### STATEMENTS OF INVENTION

According to a first aspect of the invention there is therefore provided a genetically transformed or transfected bacterial cell wherein said bacteria is a gut symbiont of an insect belonging to the Order Thysanoptera characterised in that said bacterial cell is transformed to express dsRNA against at least a part of tubulin gene or at least a part of elongation factor gene of the insect.

In a preferred embodiment of the invention said bacterial cell is transformed to express dsRNA against at least a part of tubulin alpha-1 chain gene or at least a part of elongation factor 1-alpha gene of the insect.

Reference herein to tubulin alpha-1 chain gene is to the gene having accession number GT305545 (GenBank; NCBI). and reference herein to elongation factor 1-alpha gene is to the gene having accession number GT303726 (GenBank; NCBI).

In a preferred embodiment of the invention, said dsRNA comprises a strand of RNA that shares 50% complementarity to at least a part of said tubulin gene or at least a part of elongation factor gene. It is preferred that said dsRNA comprises a strand of RNA that shares at least 75% complementarity to at least one of said genes of said insect and, in increasing order of preference, at least 80%, 85%, 90%, 91%, 92%, 93%, 94%, 95%, 96%, 97%, 98% or 99% A complementarity to at least one of said target genes of said insect.

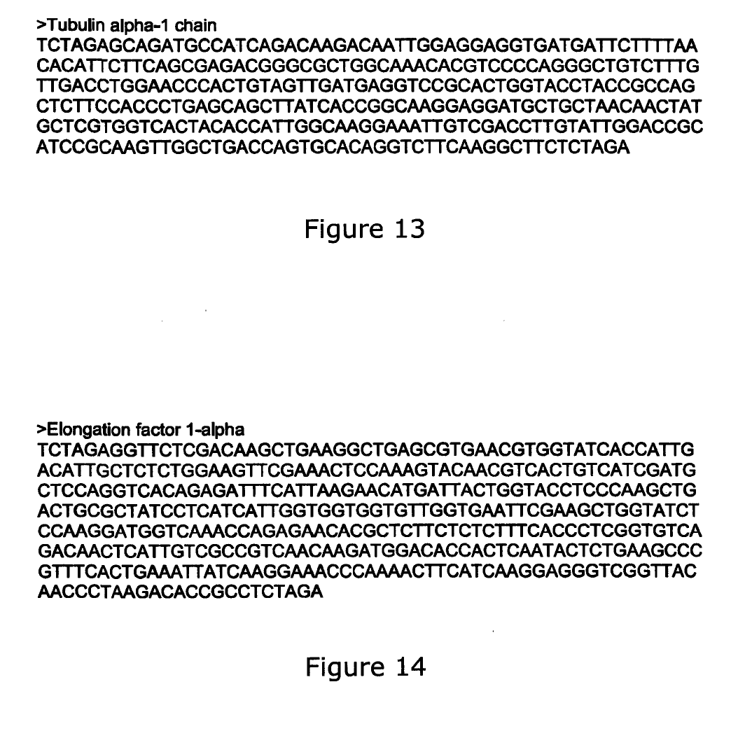

More ideally still said dsRNA active against said tubulin alpha-1 chain gene or said elongation factor 1-alpha gene of the insect is complementary to at least a part of the sequence structure shown in FIG. 13 or 14, respectively. It is preferred that said dsRNA comprises a strand of RNA that shares at least 75% complementarity to at least a part of the sequence structure shown in FIG. 13 or 14 and, in increasing order of preference, at least 80%, 85%, 90%, 91%, 92%, 93%, 94%, 95%, 96%, 97%, 98% or 99% complementarity to at least a part of the sequence structure shown in FIG. 13 or 14.

Most ideally said bacteria is related to the genus Pantoea, which is a gut symbiont of said insect, ideally, said bacteria is BFo2, with a genome sequence having accession number SAGS00000000 (GenBank, NCBI). Alternatively, said bacteria belongs to the genus Erwinia, which is a gut symbiont of said insect, ideally, said bacteria is BFo1, with a genome sequence having accession number LAGP00000000 (GenBank, NCBI).

More preferably, said insect is a member of the Thripidae family. More preferably still, said insect belongs to the genus Frankliniella such as the species Frankliniella occidentalis.

In yet a further preferred embodiment of the invention there is provided an expression vector for transforming or transfecting said bacterial cell wherein said vector comprises a nucleic acid sequence that expresses dsRNA against at least a part of said tubulin gene or at least a part of said elongation factor gene of the insect.

In a preferred embodiment of the invention said expression vector expresses dsRNA against at least a part of tubulin alpha-1 chain gene or at least a part of elongation factor 1-alpha gene of the insect.

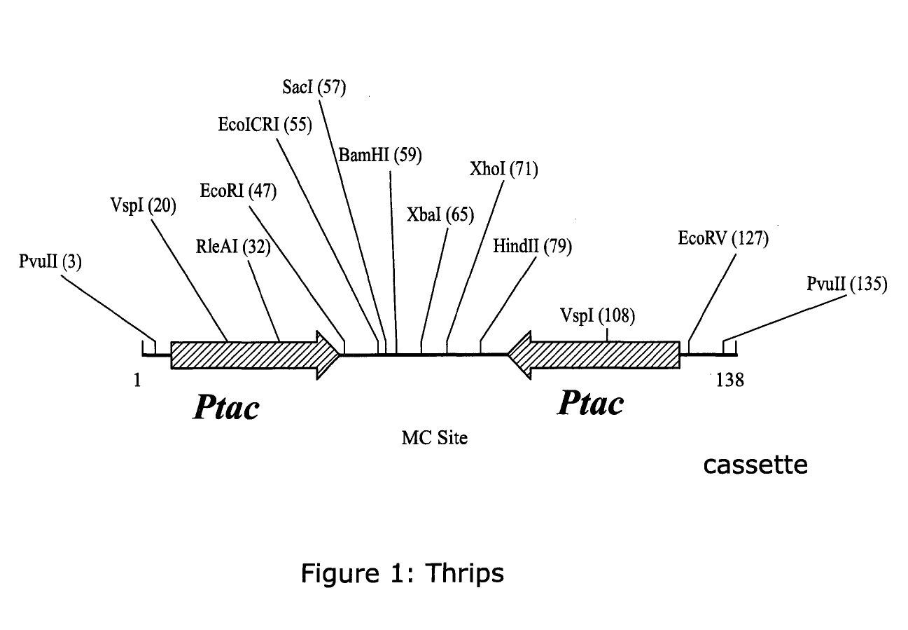

More preferably still said expression vector for transforming or transfecting said bacterial cell comprises at least one constitutive promoter, for example Ptac. Yet more preferably a plurality, such as a pair, of said promoters are provided and configured to drive transcription in a convergent manner to ensure transcription from both complementary strands of nucleic acid that expresses said dsRNA. Yet more preferably said expression vector for transforming or transfecting said bacterial cell is configured as shown in FIG. 1 i.e. configured as per the Pex-A vector. More ideally still said expression vector for transforming or transfecting said bacterial cell is pTub3 or pElong1. Advantageously, pTub3 or pElong1 comprise nucleic acid sequences that are templates for dsRNA synthesis which are flanked by convergent Ptac promoters to ensure constitutive transcription of two complementary RNA strands that hybridise to generate the desired double stranded RNA.

In a preferred embodiment of the invention, said bacterial cell is transformed or genetically modified such that recombinant DNA is stably integrated into the host cell genome. This advantageously ensures long-term target gene silencing and ensures spread in insect populations. Ideally, stable integration is achieved by way of site specific integration, typically following the use of conventional site specific integration sites. Preferably, site specific integration is achieved in the RNaseIII gene.

Those skilled in the art will be aware said insect causes a disease in plants typically by the transmission of pathogenic organism belongs to the tospoviruses including Groundnut ringspot virus or tomato spotted wilt virus.

The working of the invention is particularly effective because the insect engages in horizontal gut transfer i.e. the acquisition of gut flora bacteria by ingestion from an environmental source. Thus, working of the invention is particularly effective because said horizontal gut transfer is achieved by ingestion of faeces or frass from other insects. More ideally still, ingestion of faeces or frass is genus-specific, and believed to be species-specific, whereby the parent generation of a species transfers the genetically engineered gut symbiont to an off spring generation. In this way, said insect can acquire said transformed or transfected bacterial cell from contaminated faeces or frass in the environment, circumventing the need for insect handling and associated mortality. Furthermore, advantageously, this permits horizontal transfer of dsRNA mediating RNAi throughout at least one insect colony and, typically, many insect colonies.

Additionally, or alternatively to the site specific integration mentioned above, in yet a further preferred embodiment of the invention, said transformed or transfected bacterial cell is also genetically engineered such that it does not produce functional RNA degrading proteins, including but not limited to, RNase III. Advantageously, this minimises the risk of enzymatic degradation of said dsRNA encoded by the transformed bacterial cell. Ideally, this is by deleting the whole or a part of native RNase III gene and, ideally replacing it with an antibiotic resistant gene, for example, an apramycin resistance gene.

Advantageously, targeting of the tubulin alpha-1 chain gene or elongation factor 1-alpha gene results in early mortality of said insect. In this way, the insect's capacity to both transmit a pathogenic micro-organism is reduced and damage to foliage due to feeding or egg laying is limited.

In use, the insect acquires the dsRNA genetically transformed or transfected gut symbiont bacterial cell from its environment through ingestion. Said bacterial cell thereby establishes itself as a living population in the gut of the insect, wherein it divides and actively transcribes the dsRNA which it encodes. Advantageously, this therefore mediates RNAi in the insect indefinitely. The dsRNA is ideally targeted against tubulin alpha-1 chain gene or elongation factor 1-alpha leading to its modulation and more specifically, its down regulation. Given the importance of these genes the insect is suitably compromised and so killed.

According to a second aspect of the invention there is provided an insect belonging to the Order Thysanoptera characterised in that said insect comprises a genetically transformed or transfected bacterial cell wherein said bacteria is a gut symbiont of said insect and is transformed to express dsRNA against at least a part of tubulin alpha-1 chain gene or at least a part of elongation factor 1-alpha gene of the insect.

Ina preferred embodiment of the invention said bacteria is transformed or transfected to express dsRNA against at least a part of tubulin alpha-1 chain gene or at least a part of elongation factor 1-alpha gene of the insect.

According to a third aspect of the invention, there is provided the use of the afore described bacterial cell in a method for modulating expression of a target gene of an insect belonging to the Order Thysanoptera comprising:

contaminating a composition to be ingested by the insect with said bacterial cell; whereupon ingestion of said bacterial cell by said insect results in said bacterial cell colonising the gut of said insect wherein it synthesises dsRNA against at least a part of tubulin gene or at least a part of elongation factor gene to modulate said insect gene expression.

In a preferred use of said invention said bacterial cell is transformed or transfected to express dsRNA against at least a part of tubulin alpha-1 chain gene or at least a part of elongation factor 1-alpha gene of the insect.

In a preferred embodiment of the invention, said composition comprises a food source for the insect. More preferably still, said composition is the faeces or frass of said insect, or a food source of the juvenile insect such as, but not limited to, a plant source.

In a further preferred embodiment of the third aspect of the invention, modulating said target gene expression kills said insect, ideally at the larval stage, and so is an effective method of pest control or elimination.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The invention will now be described by way of example only with reference to the following figures wherein:—

FIG. 1: shows the configuration of a Thrips cassette. Converging promoters are indicated in red. Notice that Ptac promoters do not contain an operator sequence, therefore are not subject to LacI repression.

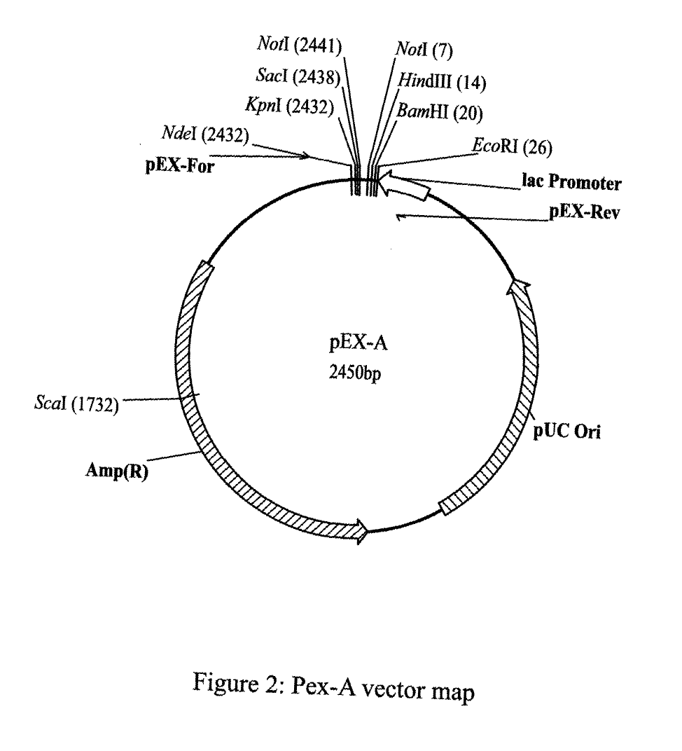

FIG. 2: shows a Pex-A vector map (Eurofins, http://www.operon.com/products/gene-synthesis/ListOfVectors.aspx).

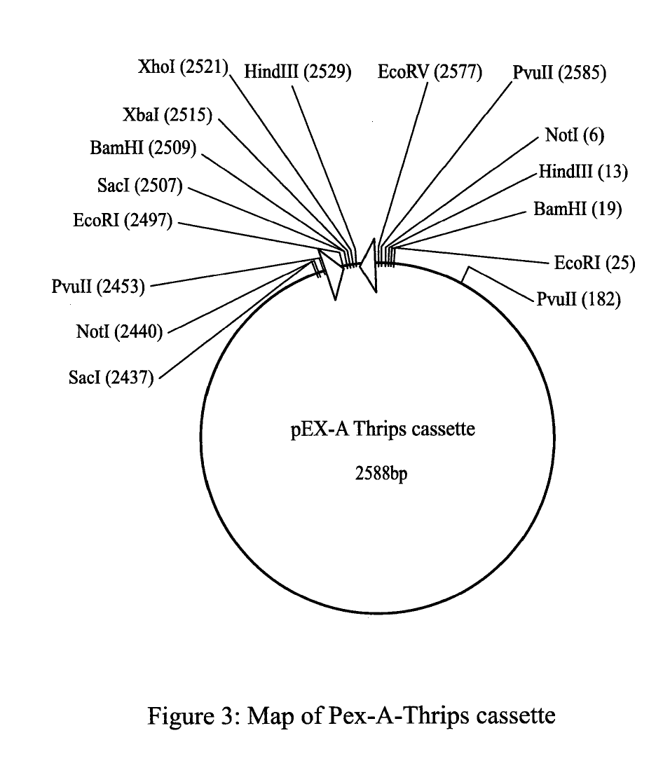

FIG. 3: shows a Map of the pEX-A-Thrips cassette.

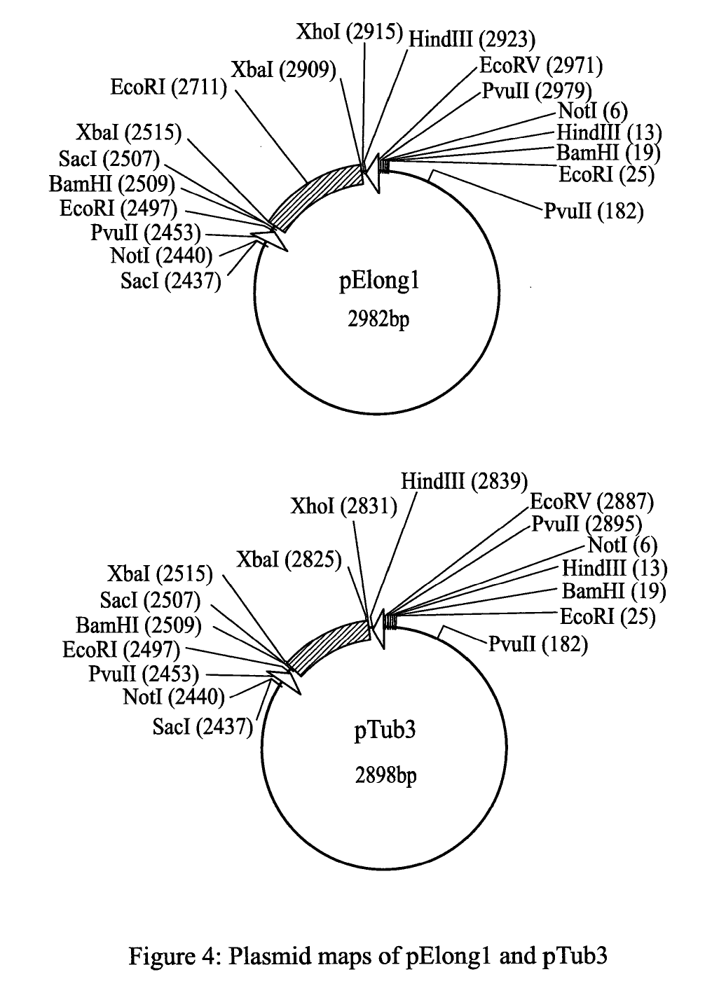

FIG. 4: shows plasmid maps of pElong1 and pTub3. Red arrows indicate Ptac promoters and blue section indicates position of dsRNA template sequence.

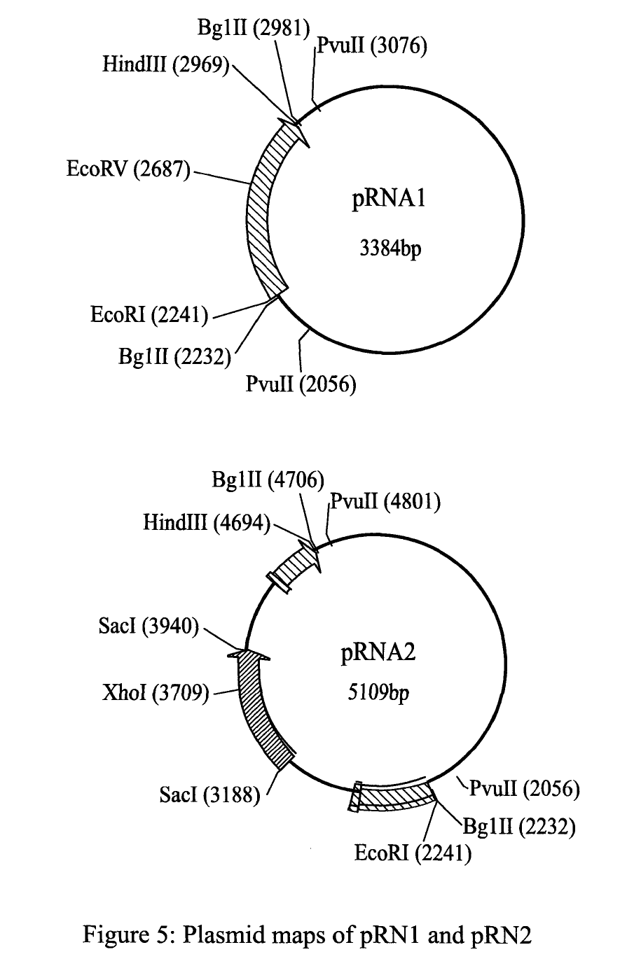

FIG. 5: shows plasmid maps of pRN1 and pRN2. The rnaseIII gene is shown in green. Light blue boxes indicate T4 transcription terminators and black arrow indicates position of the apramycin resistance gene.

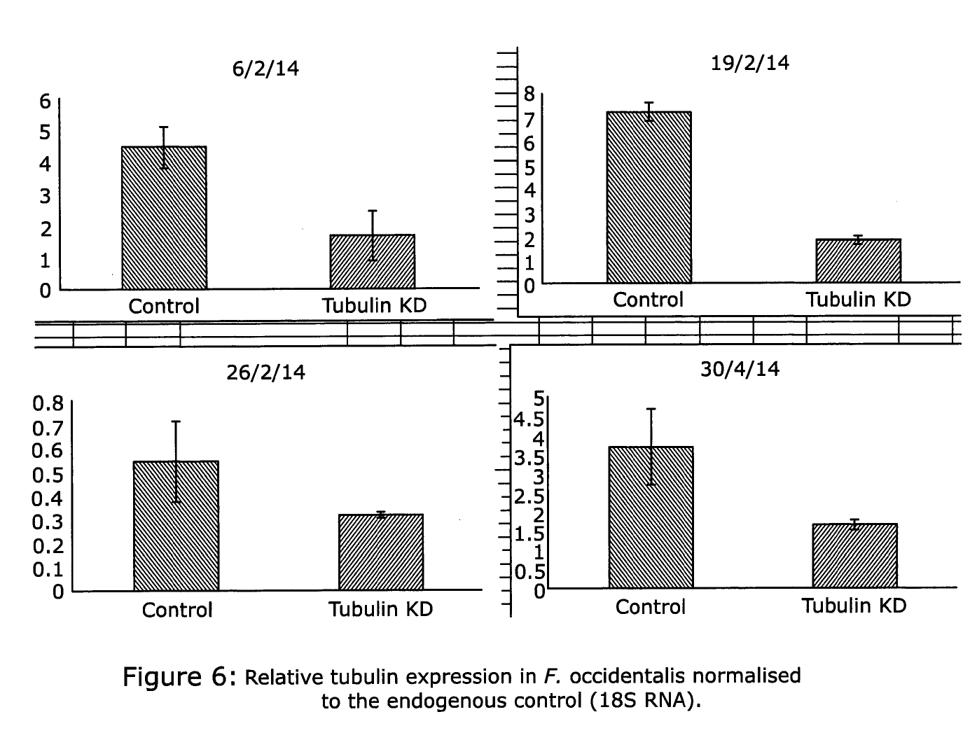

FIG. 6: shows relative tubulin expression in F. occidentalis normalised to the endogenous control (18S RNA). Bars represent mean relative transcript abundance of tubulin in juvenile insects 48 hours after being fed bacteria (BFo2) expressing double stranded agarose (control) and double stranded tubulin (Tubulin KD). Bars represent mean transcript abundance, error bars represent the standard deviation about the mean. Y axis represent relative tubulin transcript abundance normalised to 18S RNA. Each panel represents a separate experiment using a pool of approximately 22 F. occidentalis insects per treatment.

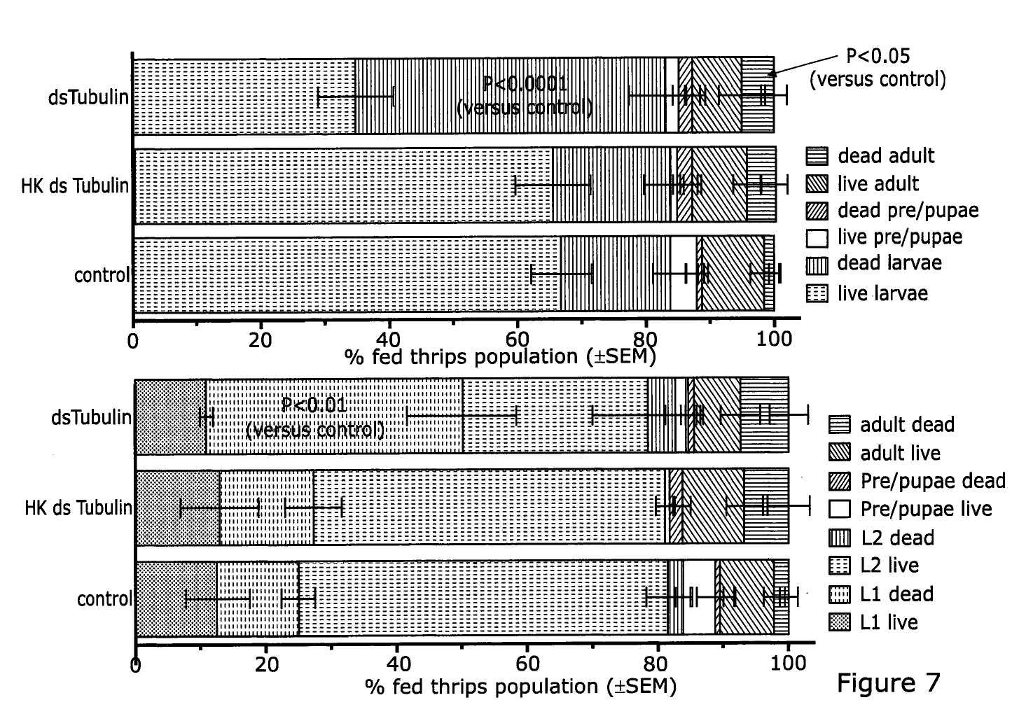

FIG. 7: shows upper panel: Composition of F. occindentalis populations following 4 days' oral exposure to modified BFo2 strains expressing control dsRNA or dsTubulin, showing significant a mortality phenotype in the larval and adult stages exposed to dsTubulin. Data compiled from 7 independent experiments; number of fed insects=234 (control), 150 (heat-killed [HK] dsTubulin), 220 (dsTubulin). Note: the pre-pupal and pupal stages of thrips are non-feeding. A highly significant tubulin knockdown mortality phenotype was observed among the thrips larvae, and particularly in the first (L1) larval stage. A small but significant mortality was also observed among the adult F. occidentalis. Heat-killed (HK) BFo2 expressing dsTubulin failed to elicit the mortality phenotype, which highlights the importance of reintroducing live dsRNA-expressing bacteria as opposed simply to pre-synthesized dsRNA. Lower panel: Additional experiments identifying the 1^{st} larval stage as most susceptible to the mortality phenotype. Data compiled from 4 independent experiments; number of fed insects=134 (control), 95 (HK dsTubulin) & 81 (dsTubulin).

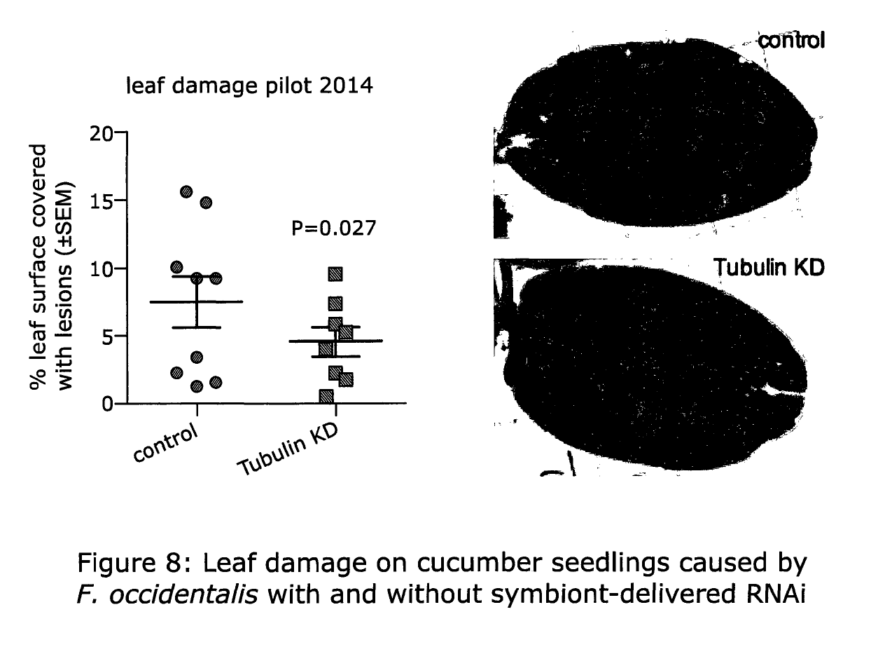

FIG. 8: shows leaf damage on cucumber seedlings caused by F. occidentalis with and without symbiont-delivered RNAi. Groups of cucumber seedlings were each exposed to WFT larvae and adults that had been orally infected with bacteria (BFo2) expressing dsAgarase RNA (control), or dsTubulin RNA (Tubulin KD). Significantly less damage occurred on plants exposed to F. occidentalis receiving the dsTubulin knockdown compared with the controls.

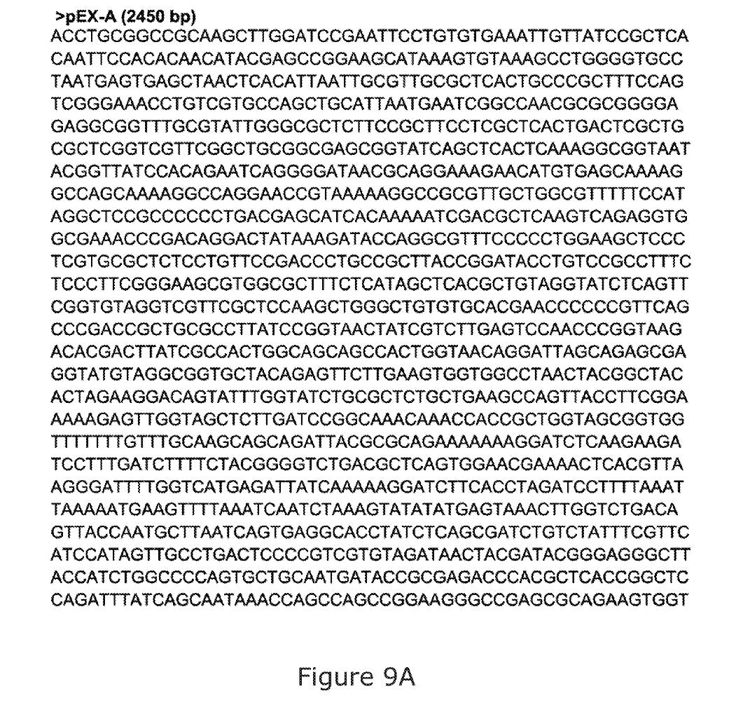

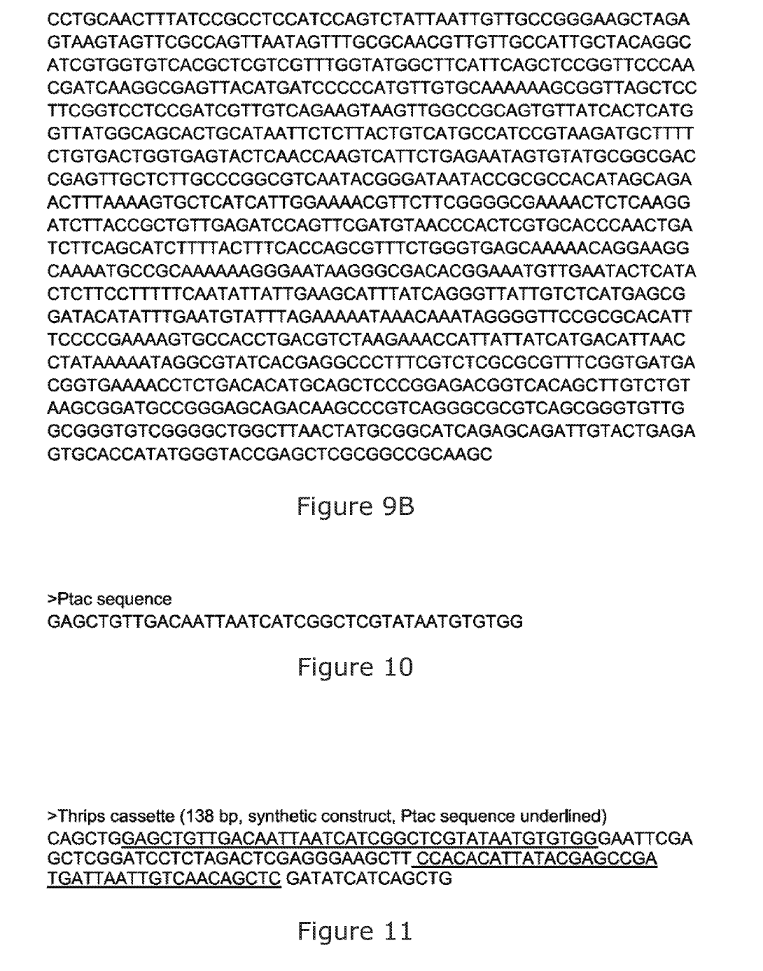

FIG. 9: shows the sequence structure of the pEX-A (2450 bp) vector.

FIG. 10: shows the sequence structure of the Ptac promoter sequence.

FIG. 11: shows the sequence structure of the Thrips cassette (138 bp, synthetic construct, Ptac sequence underlined).

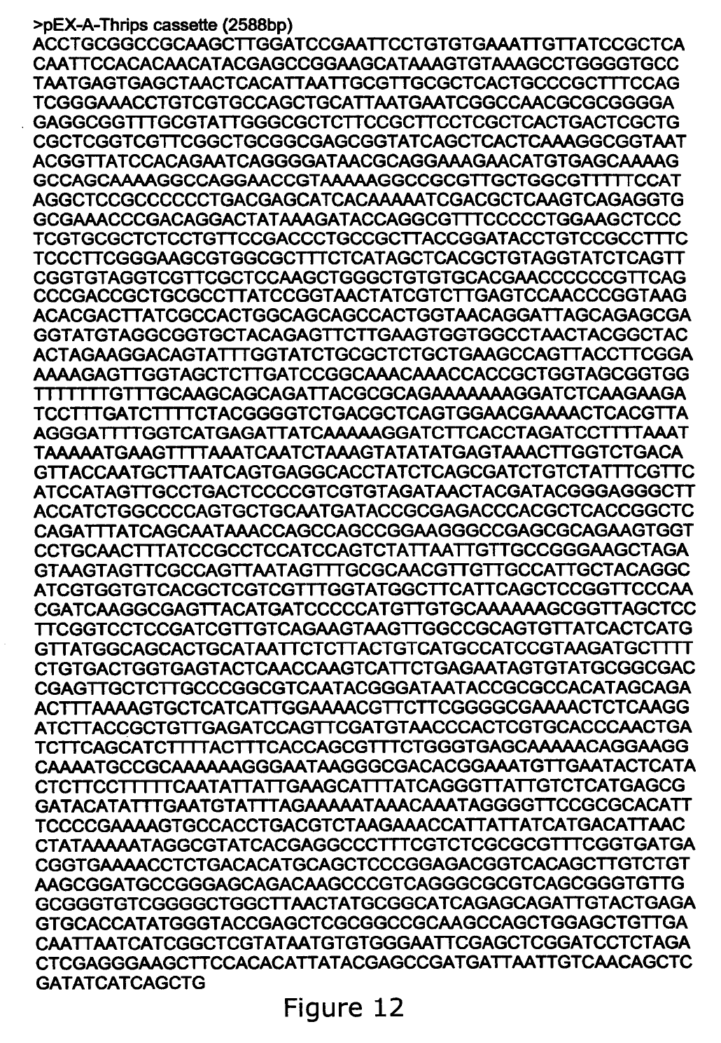

FIG. 12: shows the sequence structure of the pEX-A-Thrips cassette (2588 bp).

FIG. 13: shows the sequence structure of the Tubulin alpha-1 chain gene.

FIG. 14: shows the sequence structure of the Elongation factor 1-alpha gene.

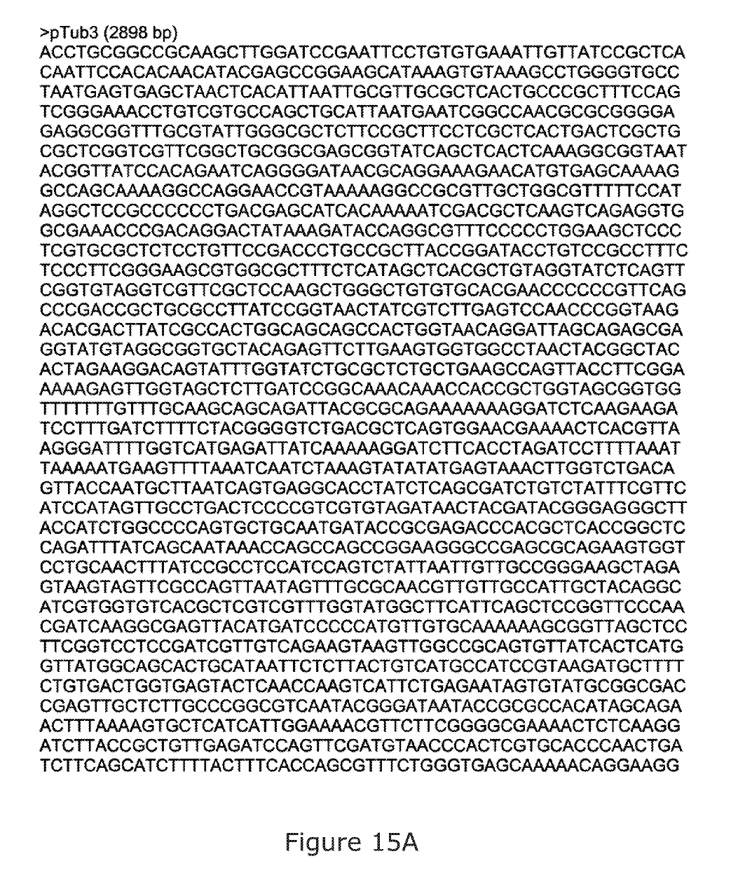

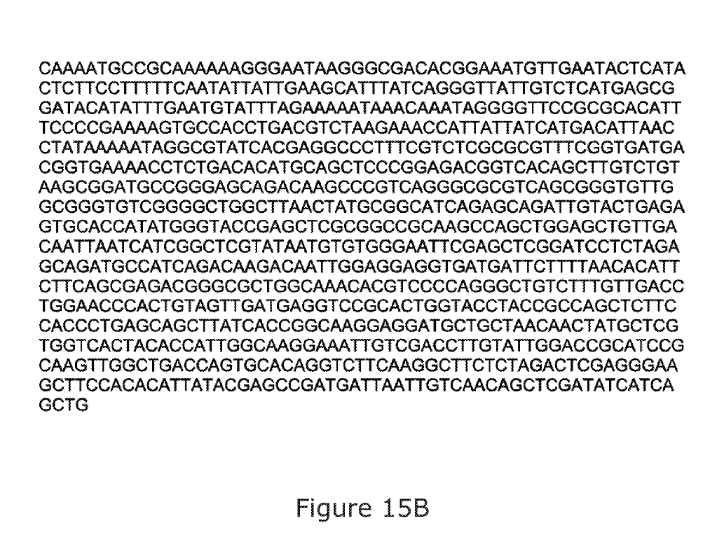

FIG. 15: shows the sequence structure of the pTub3 plasmid (2898 bp).

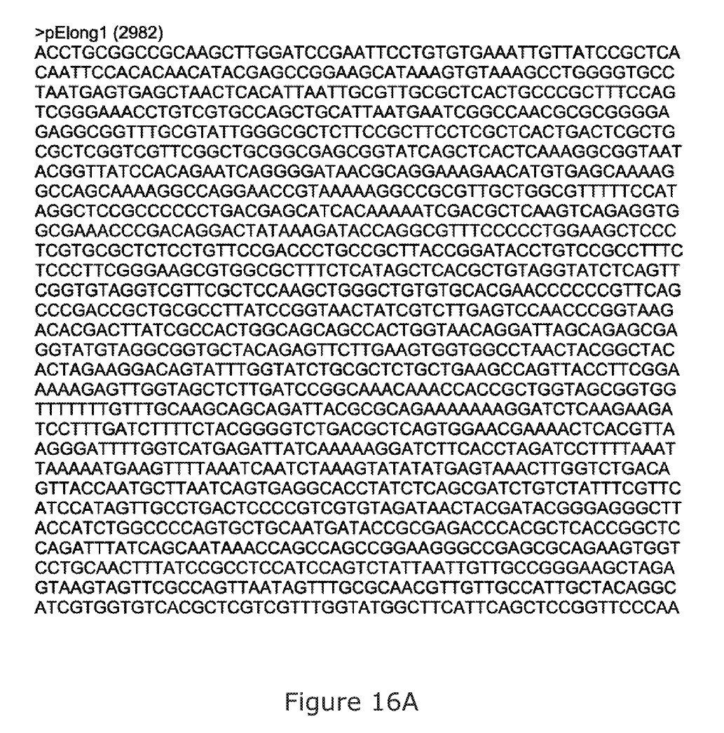

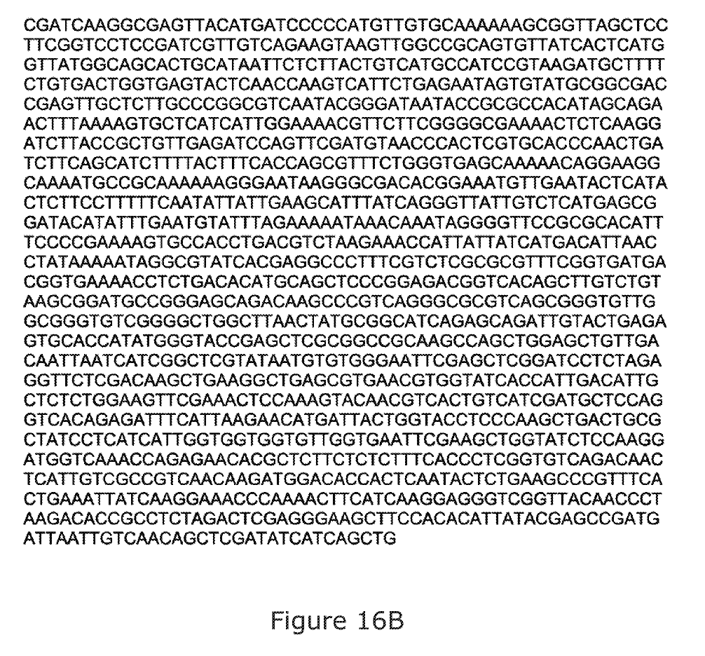

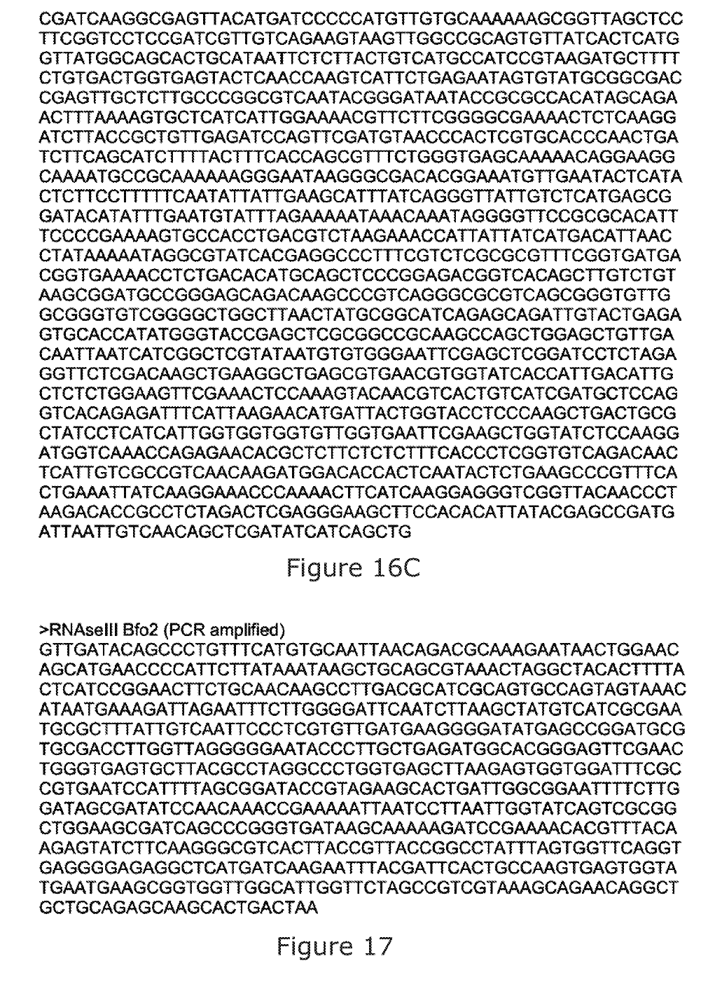

FIG. 16: shows the sequence structure of the pElong1 plasmid (2982).

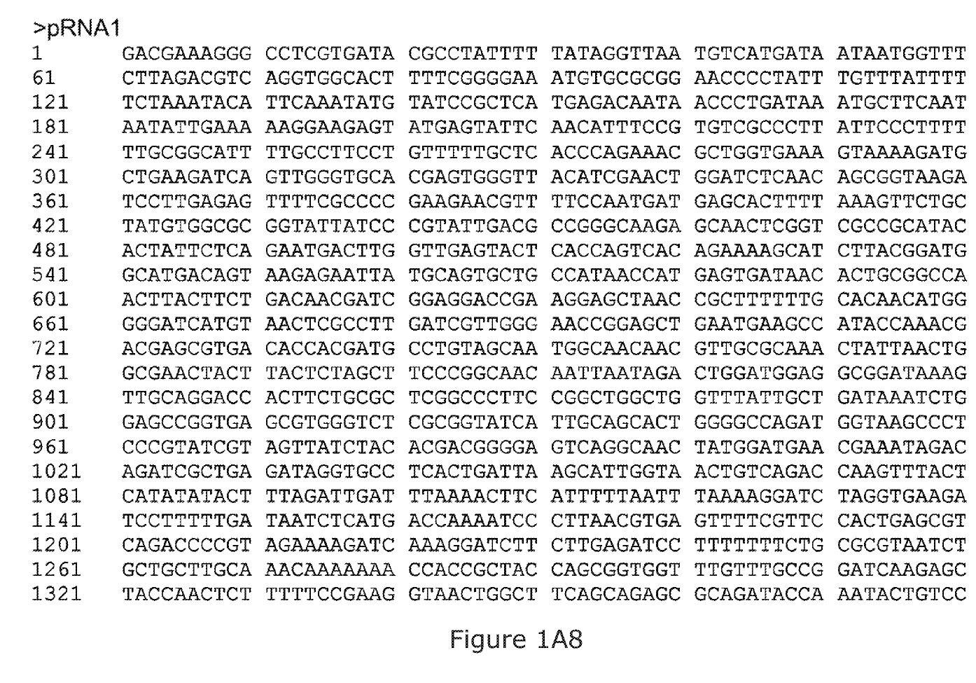

FIG. 17: shows the sequence structure of RNAseIII Bfo2 (PCR amplified).

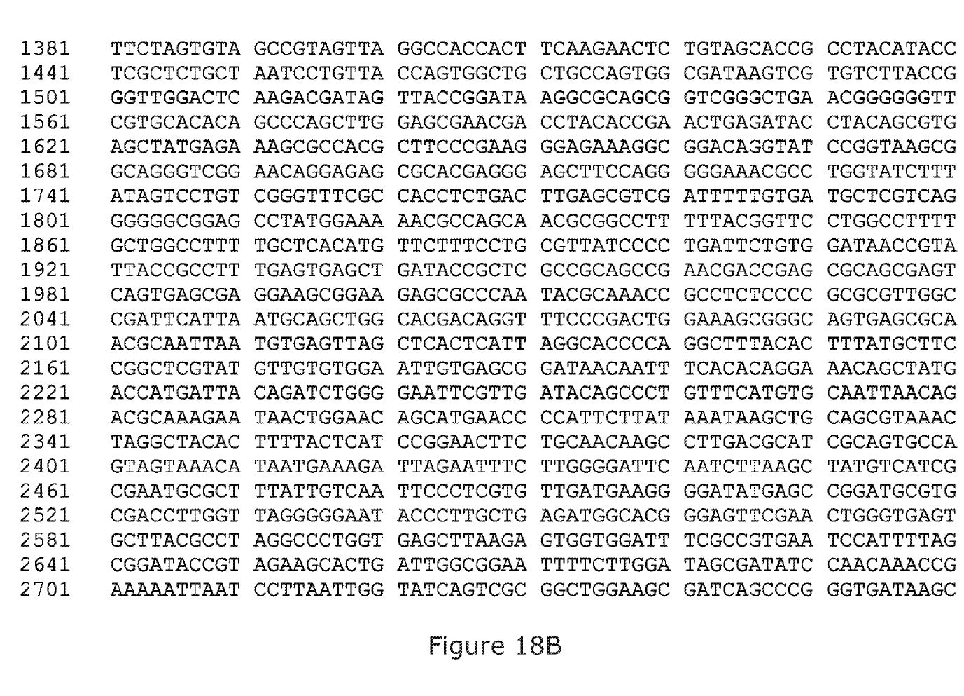

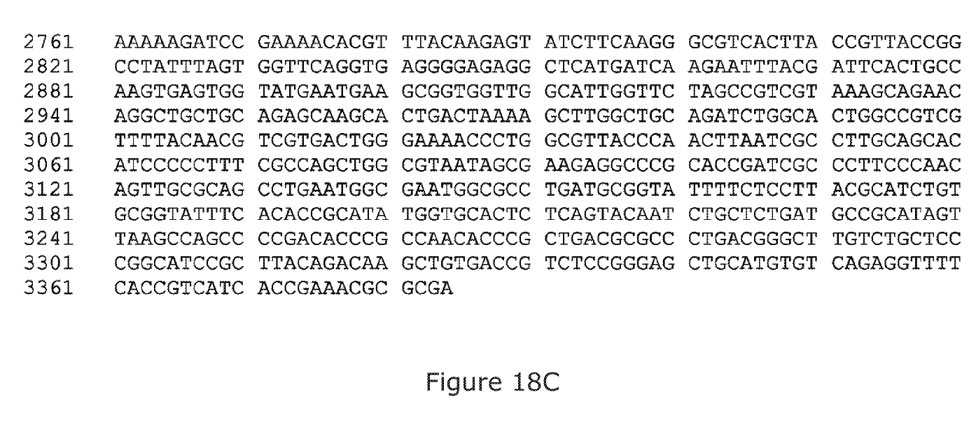

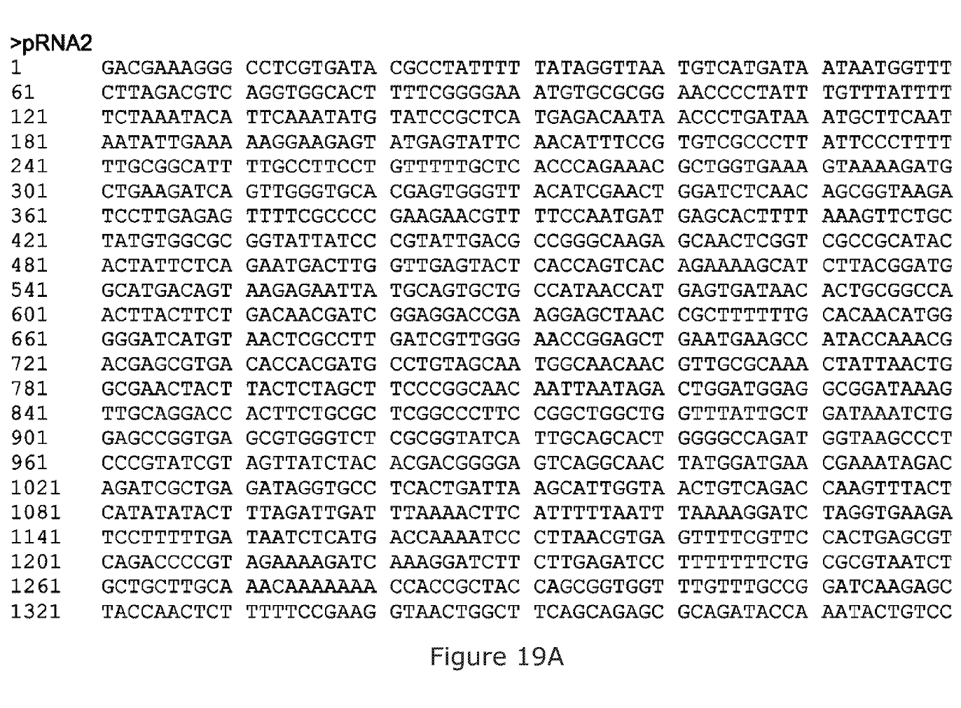

FIG. 18: shows the sequence structure of pRNA1 plasmid.

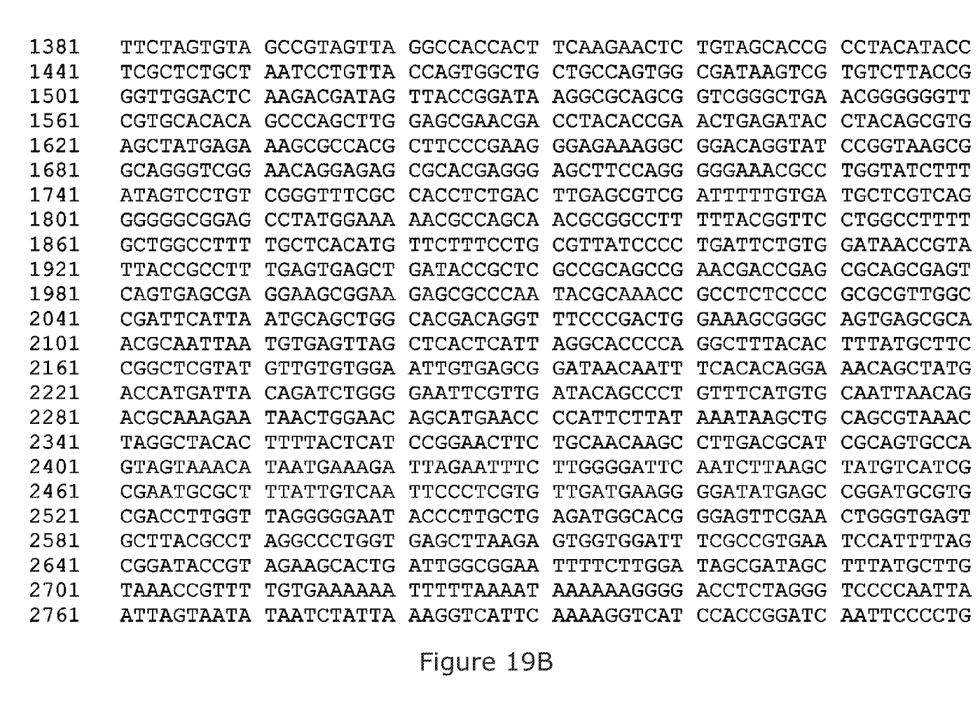

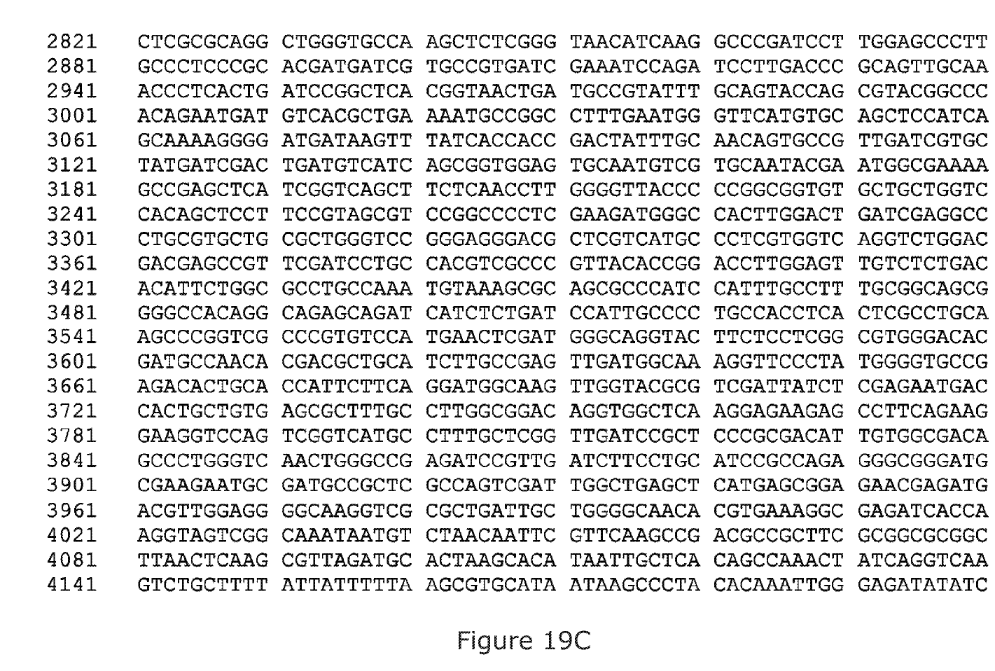

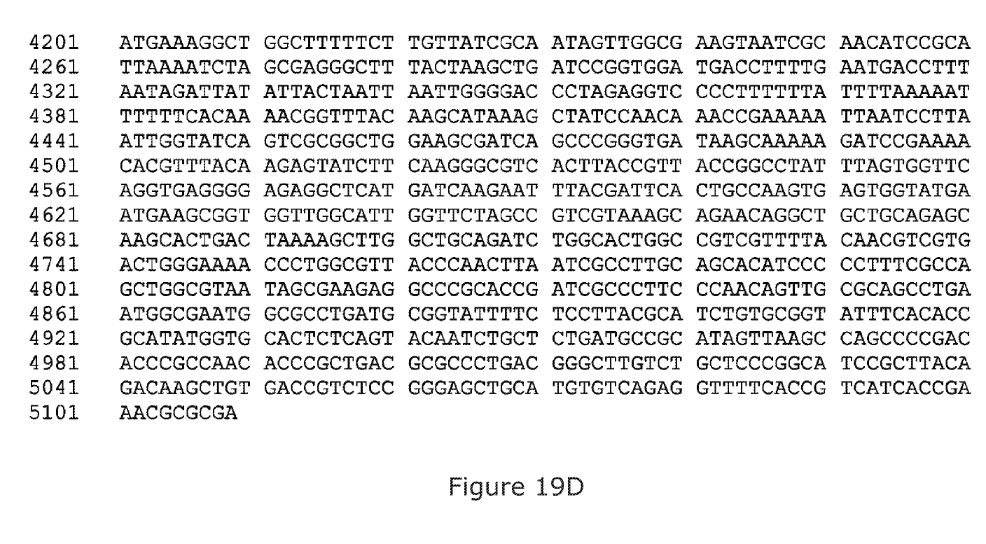

FIG. 19: shows the sequence structure of pRNA2 plasmid.

### EXAMPLE 1

The method involves establishing symbiont-mediated RNAi as a new, robust and tractable means for systemic prolonged gene silencing, suppression or knockdown in WFT, providing a vital research tool to complement the ongoing WFT genome sequencing project (https://www.hgsc.bcm.edu/western-flower-thrips-genome-project) and a potential innovative biocontrol strategy.

WFT contain two species of gut bacteria, one belonging to the genus Erwinia, named BFo1, and the second related to the genus Pantoea, named BFo2, which can also grow outside the insect host. The bacteria present in the thrips' gut are transmitted to progeny via the leaves that both adults and larvae eat and defaecate on. In second instar larvae, all thrips are infected with the bacteria, and up to 10⁵ bacterial cells are present per thrip. The bacteria attain high populations in larvae, they can be cultured on plates and are tractable for genetic manipulation. The genomes of both BFo1 and BFo2 have been sequenced. Control of transcription is similar to that of E. coli.

Stable dsRNA synthesis is dependent on (i) inactivating bacterial RNase III, and (ii) integrating the dsRNA expression cassette into the bacterial genome. This is achieved by engineering the expression cassette into a suitable plasmid and deleting the BFo2 RNase III gene. Recombinants are isolated in which the native RNase III gene is deleted and replaced by an apramycin resistance gene. Plasmids are introduced expressing dsRNA (optimised for RNAi) to target the following WFT genes: tubulin alpha-1 chain (accession number GT305545; GenBank, NCBI); or elongation factor 1-alpha (accession number GT303726; GenBank, NCBI). Knockdown of either of the two target genes severely disables larvae and indicates that the technology is effective against WFT. Stable dsRNA synthesis for each cassette is determined for each recombinant bacterial strain by Q-RTPCR.

Experimental infections were performed in different developmental stages of WFTs by feeding WFTs on recombinant dsRNA-expressing Erwinia TAC strains resuspended in an artificial feeding mixture. Thrips were membrane-fed on this mixture as the only food source for 2-4 days. Dye was included in the mixture to non-invasively identify WFTs that had fed. Bacterial growth and viability in the feeding mixture was confirmed at the beginning and end of each experiment by culturing on selective media. The gut contents of WFTs was also cultured on selective media to verify the viability and population of ingested recombinant BFo2 bacteria. Retention of the symbiotic characteristics of the bacteria was assessed by following WFT development in repopulated insects expressing dsAgarase (negative control) and dsTubulin, correlating these measurements with the presence of recombinant bacteria in the gut and by Q-RTPCR of Tubulin mRNA present in RNA preparations from the insects.

Insects populated with BFo2 strains expressing dsRNA targeting the insect tubulin genes were compared with the insects populated with bacteria expressing the negative control dsRNA. Data for insect mortality was determined in each case. These data were correlated with Q-RTPCR assays to measure the abundance of m RNA of the respective target genes.

This novel RNAi strategy can prevent infection of plants by tospoviruses (by killing juvenile insects before they can fly, by targeting vector competence genes of the insect, for example encoding an attachment protein for the virus [Kikkert M., Meurs C., van de Wetering F., Dormüller S., Peters D., Kormelink R., Goldbach R., 1998. Phytopathology 88: 63-69] and/or viral gene expression), and provide a platform for devising an effective crop protection strategy using the recombinant bacteria as a biopesticide.

Bacteria typically express an enzyme, RNaseIII, which specifically degrades dsRNA. Indeed we have established that dsRNA is unstable after it is expressed in BFo2. To circumvent this problem, we have engineered a BFo2 mutant strain in which the gene encoding RNaseIII is disrupted and which stably expresses dsRNA.

### Materials and Procedure

### Bacterial Strains and Media

Cloning procedures were performed in E. coli JM109. Disruption of the RNaseIII gene was performed in BFo2 containing pIJ790 to allow Lambda red-mediated recombination (Gust B, Challis G L, Fowler K, Kieser T, Chater K F (2003) Proc Natl Acad Sci USA. February 18; 100(4):1541-6)). Culturing of E. coli strains was as recommended (Sambrook and Russell (2001). Molecular Cloning: A Laboratory Manual (3rd ed.). Cold Spring Harbor Laboratory Press. ISBN 978-0-87969-577-4). BFo2 was grown at 30° C. in liquid culture (LB, with shaking) and on the surface of L agar plates. The identity of the RNaseIII disruption mutant was confirmed by PCR.

dsRNA Expression System

### Construction of Expression Vector to Drive Constitutive Expression of Double Stranded RNA in Bfo2.

A 138 bp synthetic expression cassette (Eurofins), containing several restriction sites flanked by two copies of the modified constitutive promoter Ptac was used. The promoter sequences were designed to drive transcription in a convergent manner (FIG. 1), to ensure transcription from both complementary strands of DNA fragments sub-cloned in the MC site. The synthetic expression cassette was cloned between the Notl sites of the ampicillin resistant plasmid pEX-A (Eurofins, FIG. 2), generating plasmid pEX-A-Thrips cassette.

Deqor (Henschel et al, 2004, Nucleic Acids Research, 32 (Web Server issue): W113-20) was used to help predict F. occidentalis genes used as target candidates for double stranded RNA mediated interference. The sequences deemed suitable candidates were obtained by synthesis (Eurofins), flanked by Xbal sites. These DNA fragments were provided as inserts cloned in pEX-A or pCR2 vectors (Table 2).

The DNA fragments to be used as templates for dsRNA synthesis were sub-cloned into the Xbal site of pEX-A-Thrips cassette, and therefore flanked by convergent Ptac promoters to ensure constitutive transcription of two complementary RNA strands that would hybridise to generate the desired double stranded RNA. The resulting in plasmids were pElong1 and pTub3. These constructs were verified by restriction and DNA sequencing (FIG. 4).

### Generation of a BFo2 Disruption Mutant.

The RNaseIII gene was PCR amplified from wild-type BFo2 using primers RIIIBfo2F1 and RIIIBfo2R1 (Table 1). The product (759 bp in length) was digested with EcoRI/HindIII and ligated into pIJ2925 previously digested with EcoRI/HindIII generating plasmid pRNA1. Disruption of this copy of the RNaseIII gene was achieved by EcoRV digestion of pRNA1 and insertion of the apramycin resistance gene (flanked by T4 transcription terminators) excised from plasmid pQM5062 by HindIII restriction digest and blunt ending using T4 DNA polymerase in the presence of 1 mM dNTPs giving rise to pRNA2 (FIG. 5). To facilitate lambda-red mediated transformation, electrocompetent BFo2 cells were created as recommended for E. coli (Sambrook and Russell (2001). Molecular Cloning: A Laboratory Manual (3rd ed.). Cold Spring Harbor Laboratory Press. ISBN 978-0-87969-577-4) and transformed by electroporation with plasmid pIJ790 (Gust B, Challis G L, Fowler K, Kieser T, Chater K F (2003) Proc Natl Acad Sci USA. February 18; 100(4):1541-6))—creating strain BFo2/pIJ790. The disrupted RNaseIII gene was excised from pRNA2 by digesting BglII and, after gel purification was introduced into electrocompetant BFo2/pIJ790 by electroporation. Double cross-over transformants were selected by replica plating on selective media containing ampicillin or apramycin. Viable colonies were tested for the presence of the disruption by colony PCR using the primers BFo2RNasetestF and BFo2RNasetestR. All plasmids were confirmed by restriction digestion and sequencing.

Testing Functionality of dsRNA Expression System

Overnight cultures of BFo2 containing the dsRNA expression plasmids were pelleted by centrifugation (13,000×g for 10 min) and mixed with equal volumes of RNA protect (Qiagen). Total RNA was isolated using the RNeasy mini RNA isolation kit (Qiagen). Genomic DNA was digested using on-column DNAse I treatment for 45 minutes at room temperature. Strand specific reverse transcription was performed on 1 ug of total RNA using the iscript select reverse transcription kit (Biorad) including the gene specific primer enhancer (GSP) and one of the following gene specific primer pairs: tubulin—TubFoF1 and TubFoR1, elongation factor—EFFoF1 and EFFoR1, (sequences shown in table 3). cDNAs were diluted 1/3 in nuclease free water (supplier). Strand-specific Real Time amplification was performed in triplicate using Sybr-Green Supermix (Biorad) and both gene specific primers listed in table 1. No template controls (NTC, non-reverse transcription) were used to assess the extent of genomic DNA carry over.

Infection of Thrips with BFo2 Strains

Overnight cultures of BFo2 containing the dsRNA expression plasmids were pelleted by centrifugation (3,000×g for 2 min) and washed by resuspension in Luria broth, then resuspended to 5×10⁶/ml in an artificial feeding mixture (20% (v/v) Luria broth, 2.4% (w/v) sucrose, 0.32% (w/v) NaCl and 0.03% (w/v) methylene blue). Franliniella occidentalis of all developmental stages were membrane-fed on the feeding mixture as the only food source for 2-4 days from an inverted Bijou bottle reservoir covered by stretched Parafilm. Methylene blue was included to non-invasively identify WFTs that had fed (the blue colour in the gut being visible through the insect cuticle under low-power magnification in WFTs anaesthetised by CO₂). Bacterial growth and viability in the feeding mixture was confirmed at the beginning and end of each experiment by culturing on LB agar supplemented with 1.5% (w/v) sucrose and the appropriate selective antibiotic (apramycin for BFo2 expressing dsAgarase; apramycin and ampicillin for BFo2 expressing dsTubulin). The dyed gut contents of randomly-selected, surface-sterilized WFTs were also cultured on selective media to verify the viability and population of ingested BFo2 strains. Additional controls were prepared using overnight cultures of BFo2 expressing dsTubulin, which were heat-killed by incubation at 65° C. for 20 mins prior to incorporation into feeding mixtures as above.

### Efficacy of RNAi in Infected Insects

Juvenile thrips (1^{st} and 2^{nd} instar) were sampled 48 hours after infection with recombinant BFo2, RNA extracted and levels of tubulin alpha1 mRNA quantified (FIG. 6). Total RNA was isolated from pools of approximately 22 juvenile insects using the ZR Tissue and insect RNA microprep kit (Zymo Research). On-column DNase treatment was performed at room temperature using RNase free DNAse (Qiagen). For each isolation, 500 ng of RNA was reverse transcribed using the iscript select synthesis kit and oligodT primers (BioRad). Q RT-PCRs were performed in triplicate using Sybr Green Supermix (BioRad). Serial dilutions of pooled cDNA were performed in duplicate and used to generate standard curves and to test the efficiency of the q RT PCR reaction. Relative transcript abundance of alpha tubulin was calculated by normalising to 18S. All Q RT PCR primers are listed in table 3.

### Mortality of Infected Insects

Frankliniella occidentalis populations were reared on chrysanthemum plants and runner beans ad libitum at 70-80% relative humidity, 26-27° C., with a light:dark cycle of 14:10 hours respectively. Sample WFT populations containing all developmental stages of F. occidentalis were orally infected with recombinant BFo2 expressing dsAgarose RNA (control) and dsTubulin, and monitored for a knockdown phenotype after 4 days. An additional control was included which involved feeding of heat-killed BFo2 expressing dsTubulin.

### Plant Protection

Groups of three 15-day-old cucumber seedlings were each exposed to 50 larvae and 15 adult female F. occidentalis that had been orally infected with BFo2 expressing dsAgarase RNA (control), or dsTubulin RNA (Tubulin KD). After 5 days, the percentage of the leaf surface that was covered with lesions was assessed by Assess 2.0 image analysis software for plant disease quantification (Lamari, Amer Phytopathological Society; 2008; http://www.apsnet.org/press/assess); (FIG. 5).

TABLE 1
Strains and plasmids.

| | genotype/comments | Source |
|---|---|---|
| Strains | | |
| E. coli | F′traD36 proA⁺B⁺laclqΔ(lacZ)M15/Δ | Yanish-Perron et al., |
| JM109 | (lac-proAB) glnV44 e14-gyrA96 | 1985 |
| | recA1relA1endA1 thi hsdR17 | |
| Plasmids | | |
| pIJ2925 | bla, lacZ | Kieser et al., 2000 |
| pRNA1 | pIJ2925 containing ~759 bp BFo2 | This study |
| | rnaseIII PCR product | |
| pRNA2 | pIJ2925 containing apramycin disrupted | This study |
| | BFo2 rnaseIII PCR product | |
| pIJ790 | λ-RED (gam, bet, exo), cat, araC, | Gust et al., 2003 |
| | rep101ts | |
| pQM5062 | pMOD + Tn5062, Ampicillin^{R} and | Bishop et al., 2004 |
| | Apramycin^{R} | |

TABLE 2
Gene targets used to generate synthetic DNA fragments to be used as
template for dsRNA synthesis.

| | | Length of |
|---|---|---|
| | | synthetic |
| Target gene (Genebank) | Plasmid name | sequence |
| Elongation factor 1A | pEX-A-Elongation factor 1A | 400 bp |
| (GT303726.1) | | |
| Tubulin Alpha-1 chain | pEX-A-tubulin alpha-1 | 316 bp |
| (GT305545.1) | | |

TABLE 3
Oligonucleotides used for PCR and Q RT-PCR.

| Oligo- | |
|---|---|
| nucleotides | |
| RIIIBfo2F1 | TTAGAATTCGTTGATACAGCCCTGTTTCATGTGC |
| RIIIBfo2R1 | TTTAAGCTTTTAGTCAGTGCTTGCTCTGCAGC |
| BFo2RNasetestf | AGC GGA TAC CGT AGA AGC AC |
| BFo2RNasetestR | TAG GCC GGT AAC GGT AAG TG |
| TubFoF1 | AGG ATG CTG CTA ACA ACT A |
| TubFoR1 | GAT GCG GTC CAA TAC AAG |
| EFFoF1 | CTC CAG GTC ACA GAG ATT |
| EFFoR1 | CAC CAA TGA TGA GGA TAG C |
| Fo18SF | GAA GGA TTG ACA GAT TGA |
| Fo18SR | TAG AGT CTC GTT CGT TAT |
| FoTubF | TTG AAG AAG CAT CCT AAC |
| FoTubR | TTG AGA AGT AGT TGA GAT |

1. A genetically transformed or transfected bacterial cell wherein said bacteria is a gut symbiont of an insect belonging to the Order Thysanoptera characterised in that said bacterial cell is transformed or transfected with nucleic acid to express dsRNA against at least a part of tubulin gene or at least a part of elongation factor gene of the insect.

2. The genetically transformed or transfected bacterial cell according to claim 1 wherein said dsRNA is against at least a part of tubulin alpha-1 chain gene or at least a part of elongation factor 1-alpha gene of the insect.

3. The genetically transformed or transfected bacterial cell according to claim 1, wherein said dsRNA comprises a strand of RNA that shares 50% complementarity to at least one of said genes.

4. The genetically transformed or transfected bacterial cell according to claim 3, wherein said dsRNA comprises a strand of RNA that shares at least 75%, 80%, 85%, 90%, 91%, 92%, 93%, 94%, 95%, 96%, 97%, 98% or 99% complementarity to at least one of said genes.

5. The genetically transformed or transfected bacterial cell according to claim 3, wherein said dsRNA active against said tubulin gene or said elongation factor gene of the insect is complementary to at least a part of the sequence of SEQ ID NO: 17 or 18.

6. The genetically transformed or transfected bacterial cell according to claim 3, wherein dsRNA comprises a strand of RNA that shares at least 75% 80%, 85%, 90%, 91%, 92%, 93%, 94%, 95%, 96%, 97%, 98% or 99% complementarity to ata part of the sequence of SEQ ID NO: 17 or 18.

7. The genetically transformed or transfected bacterial cell of claim 1, wherein said insect belongs to the Thripidae family.

8. The genetically transformed or transfected bacterial cell according to claim 7 wherein said insect belongs to the genus Frankliniella.

9. The genetically transformed or transfected bacterial cell according to claim 8 wherein said insect belongs to the species Frankliniella occidentalis.

10. The genetically transformed or transfected bacterial cell of claim 1, wherein said bacteria is a gut symbiont related to bacteria of the genus Pantoea; or an Erwinia species gut symbiont.

11. The genetically transformed or transfected bacterial cell according to claim 10 wherein said bacteria is BFo2 or BFo1.

12. An expression vector for transforming or transfecting a bacterial cell that is a gut symbiont of an insect belonging to the Order Thysanoptera wherein said vector comprises a nucleic acid sequence that expresses dsRNA against at least a part of tubulin gene or at least a part of elongation factor gene of the insect.

13. The expression vector according to claim 12 wherein said dsRNA is against at least a part of tubulin alpha-1 chain gene or at least a part of elongation factor 1-alpha gene of the insect.

14. The expression vector according to claim 12, wherein said vector comprises at least one constitutive promoter.

15. The expression vector according to claim 14 wherein said constitutive promoter is Ptac.

16. The expression vector according to claim 14, wherein said vector comprises a pair of said promoters.

17. The expression vector according to claim 16 wherein said pair of promoters are configured to drive transcription in a convergent manner.

18. The expression vector according to claim 12, wherein said vector is selected from the group comprising Pex A, pTub3, pElong1, pRN1, and pRN2.

19. An insect belonging to the Order Thysanoptera characterised in that said insect comprises a genetically transformed or transfected bacterial cell wherein said bacteria is a gut symbiont of said insect and is transformed to express dsRNA against at least a part of tubulin gene or at least a part of elongation factor gene of the insect.

20. The insect according to claim 19 wherein said dsRNA is against at least a part of tubulin alpha-1 chain gene or at least a part of elongation factor 1-alpha of the insect.

21. (canceled)

22. (canceled)

23. A method for modulating the expression of a target gene of an insect belonging to the Order Thysanoptera comprising:
contaminating a composition to be ingested by the insect with a bacterial cell according to claim 1;
whereupon ingestion of said bacterial cell by said insect results in said bacterial cell colonising the gut of said insect wherein it synthesises dsRNA against at least a part of tubulin gene or at least a part of elongation factor gene to modulate said insect gene expression.

24. The method according to claim 23 wherein said dsRNA is against at least a part of tubulin alpha-1 chain gene or at least a part of elongation factor 1-alpha of the insect.

25. The method according to claim 23, wherein said bacterial cell is transformed or genetically modified such that recombinant DNA is stably integrated into the host cell genome.

26. The method of claim 25 wherein said nucleic acid or recombinant DNA is stably integrated in the RNaseIII gene.

27. The method according to claim 23, wherein modulating the expression of a target gene causes insect death, prevents transmission of a pathogenic organism, or both.

28. The method according to claim 27, wherein said insect death occurs at the larval stage.

29. (canceled)

30. The method according to claim 27, wherein said pathogenic organism is a tospovirus.

31. The method according to claim 23, wherein said composition comprises a food source for the insect, a plant, faeces or frass.

32. (canceled)
